(19) United States
(12) Patent Application Publication
(10) Pub. No.: US 2019/0054829 A1
(43) Pub. Date: Feb. 21, 2019

(54) CHARGING CONTROL METHOD, ELECTRIC VEHICLE, AND CHARGING APPARATUS USING THE SAME

(71) Applicants: Hyundai Motor Company, Seoul (KR); Kia Motors Corporation, Seoul (KR)

(72) Inventors: Jae Yong Seong, Hwaseong (KR); Taek Hyun Jung, Hwaseong (KR)

(21) Appl. No.: 16/105,620

(22) Filed: Aug. 20, 2018

(30) Foreign Application Priority Data
Aug. 21, 2017 (KR) 10-2017-0105737
Jul. 26, 2018 (KR) 10-2018-0086983

(51) Int. Cl. B60L 11/18; G01R 33/00; H02J 7/02; A61N 1/372

(52) U.S. Cl. CPC B60L 11/182; G01R 33/0023; B60L 2230/10; A61N 1/37229; H02J 7/025

(57) ABSTRACT

A charging control method is provided for an electric vehicle (EV) receiving a power from a charging apparatus. The method includes detecting a signal from an implanted medical device (IMD) and determining a limitation on a charging output based on whether the signal from the IMD is detected. Information related to the limitation on the charging output is then transmitted to the charging apparatus which provides power to the electric vehicle.

### CROSS-REFERENCE TO RELATED APPLICATION

The present application claims priority to Korean Patent Applications No. 10-2017-0105737 filed on Aug. 21, 2017 and No. 10-2018-0086983 filed on Jul. 26, 2018, the entire contents of which is incorporated herein for all purposes by this reference.

### TECHNICAL FIELD

The present disclosure relates to a charging control method, and an electric vehicle (EV) and a charging control apparatus using the same, and more specifically, to a method for controlling wireless charging based on a presence of an occupant in an EV and a type of the occupant, an EV, and a control charging apparatus using the same.

### BACKGROUND

An electric vehicle (EV) charging system may be defined as a system for charging a high-voltage battery mounted within an EV using power of an energy storage device or a power grid of a commercial power source. The EV charging system may have various forms according to the type of EV. For example, the EV charging system may be classified into a conductive type using a charging cable and a non-contact wireless power transfer (WPT) type (also referred to as an ‘inductive type’).

When charging an EV wirelessly, a reception coil in a vehicle assembly (VA) mounted on the EV forms an inductive resonant coupling with a transmission coil in a group assembly (GA) located in a charging station or a charging spot, and electric power is transferred from the GA to the VA to charge the high-voltage battery of the EV through the inductive resonant coupling.

Meanwhile, in wireless charging of the non-contact WPT type, a driver of an EV or a person in the vicinity of the EV may be influenced by an electromagnetic field or an electromotive force. In particular, when a person wearing an implanted medical device (IMD) is exposed to the electronic magnetic field, the IMD may malfunction due to the electromagnetic field.

### SUMMARY

Accordingly, exemplary embodiments of the present disclosure provide a wireless charging control method. Additionally, exemplary embodiments of the present disclosure also provide an EV using the wireless charging control method and a wireless charging apparatus using the wireless charging control method.

According to exemplary embodiments of the present disclosure, a charging control method performed by an electric vehicle (EV) receiving a power form a charging apparatus may include detecting a signal from an implanted medical device (IMD); determining a limitation on a charging output according to whether the signal from the IMD is detected; transmitting information related to the limitation on the charging output to the charging apparatus; and receiving a power from the charging apparatus.

The signal from the IMD may be a radio frequency (RF) signal. The charging control method may further include activating a low frequency (LF) magnetic field; and detecting from the charging apparatus a response to the LF magnetic field. The determining of a limitation on a charging output may include determining to limit the charging output when the signal from the IMD and the response from the charging apparatus are detected.

The information related to the limitation may include information related to whether to limit the charging output and information related to a limitation ratio of the charging output. The limitation ratio of the charging output may be defined as a ratio to a typical charging power. The typical charging power may be defined according to at least one of the society of automotive engineers (SAE) J2954, the International Organization for Standardization (ISO) 19363, and the International Electrotechnical Commission (IEC) 61980.

Furthermore, in accordance with exemplary embodiments of the present disclosure, an EV may include at least one communication module, a charging module including a reception pad coupled with a transmission pad of a charging apparatus and receiving a power through the reception pad, at least one processor, and a memory configured to store at least one instruction executed by the at least one processor. Additionally, the at least one instruction may be configured to cause the at least one processor to detect a signal from an IMD; determine a limitation on a charging output according to whether the signal from the IMD is detected; and transmit information related to the limitation on the charging output to the charging apparatus.

The signal from the IMD may be an RF signal. The at least one instruction may be further configured to cause the at least one processor to activate a low frequency (LF) magnetic field; and detect from the charging apparatus a response to the LF magnetic field. The at least one instruction may be further configured to cause the at least one processor to determine to limit the charging output when the signal from the IMD and the response from the charging apparatus are detected.

The information related to the limitation may include information related to whether to limit the charging output and information related to a limitation ratio of the charging output. The limitation ratio of the charging output may be defined as a ratio to a typical charging power. The typical charging power may be defined according to at least one of the society of automotive engineers (SAE) J2954, the International Organization for Standardization (ISO) 19363, and the International Electrotechnical Commission (IEC) 61980. The at least one communication module may be configured to communicate with the charging apparatus using a RF communication scheme.

Furthermore, in accordance with exemplary embodiments of the present disclosure, a charging apparatus may include a communication module configured to communicate with an EV, a power transmission module including a transmission pad coupled with a reception pad of the EV and configured to supply a power to the EV through the transmission pad, at least one processor, and a memory configured to store at least one instruction executed by the at least one processor. Additionally, the at least one instruction may be configured to cause the at least one processor to detect a magnetic field activated by the EV; transmit a response signal to the magnetic field to the EV; receive from the EV information related to a limitation on a charging output configured according to whether an implanted medical device (IMD) is present in or around the EV; and supply a power to the EV according to the information related to the limitation on the charging output.

The information related to the limitation may include information related to whether to limit the charging output and information related to a limitation ratio of the charging output. The limitation ratio of the charging output may be defined as a ratio to a typical charging power. The typical charging power may be defined according to at least one of the society of automotive engineers (SAE) J2954, the International Organization for Standardization (ISO) 19363, and the International Electrotechnical Commission (IEC) 61980. The magnetic field activated by the EV may be an LF magnetic field, and the at least one communication module may be configured to communicate with the EV using an RF communication scheme.

According to the exemplary embodiments of the present disclosure, even when the user of the IMD is located in the vicinity of the EV or outside the EV during wirelessly charging of the EV, the user of the IMD may be protected from the electromagnetic field due to the wireless charging.

### BRIEF DESCRIPTION OF DRAWINGS

The present disclosure will become more apparent by describing in detail exemplary embodiments of the present disclosure with reference to the accompanying drawings, in which:

FIG. 1 is a conceptual diagram illustrating a concept of a wireless power transfer (WPT) to which exemplary embodiments of the present disclosure are applied;

FIG. 2 is a conceptual diagram illustrating a pacemaker as one of implantable medical devices to which exemplary embodiments of the present disclosure are applied;

FIG. 3 is a plan view illustrating EMF regions to explain EMF limits according to an exemplary embodiment of the present disclosure;

FIG. 4 is a front view illustrating EMF regions to explain EMF limits according to an exemplary embodiment of the present disclosure;

FIG. 5 is a table listing magnetic field limits of a pacemaker/IMD for each region in vehicle interior and exterior according to an exemplary embodiment of the present disclosure;

FIG. 6 is a conceptual diagram illustrating a wireless charging control system according to an exemplary embodiment of the present disclosure;

FIG. 7 is a conceptual diagram illustrating a charging control according to an exemplary embodiment of the present disclosure that may be applied when a user of an IMD is located in vicinity of a WPT system;

FIG. 8 is a diagram illustrating a frequency spectrum used for IMDs and a frequency spectrum of vehicle RF signals according to an exemplary embodiment of the present disclosure;

FIG. 9 is a conceptual diagram illustrating an IMD monitoring system according to an exemplary embodiment of the present disclosure;

FIG. 10 is a flowchart illustrating a charging control method according to an exemplary embodiment of the present disclosure;

FIG. 11 is a block diagram illustrating an electric vehicle according to an exemplary embodiment of the present disclosure;

FIG. 12 is a diagram illustrating a structure of a frame used for LF communications applicable to exemplary embodiments of the present disclosure;

FIG. 13 is a diagram illustrating a structure of a frame used for RF communications applicable to exemplary embodiments of the present disclosure;

FIG. 14 is a diagram illustrating a timing cycle of a pacemaker applicable to exemplary embodiments of the present disclosure; and

FIG. 15 is a block diagram illustrating a charging apparatus according to an exemplary embodiment of the present disclosure.

It should be understood that the above-referenced drawings are not necessarily to scale, presenting a somewhat simplified representation of various according to an exemplary embodiment of the present disclosure features illustrative of the basic principles of the disclosure. The specific design features of the present disclosure, including, for example, specific dimensions, orientations, locations, and shapes, will be determined in part by the particular intended application and use environment.

### DETAILED DESCRIPTION

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

Although exemplary embodiment is described as using a plurality of units to perform the exemplary process, it is understood that the exemplary processes may also be performed by one or plurality of modules. Additionally, it is understood that the term controller/control unit refers to a hardware device that includes a memory and a processor. The memory is configured to store the modules and the processor is specifically configured to execute said modules to perform one or more processes which are described further below.

Furthermore, control logic of the present disclosure may be embodied as non-transitory computer readable media on a computer readable medium containing executable program instructions executed by a processor, controller/control unit or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Exemplary embodiments of the present disclosure are disclosed herein. However, specific structural and functional details disclosed herein are merely representative for purposes of describing exemplary embodiments of the present disclosure, however, exemplary embodiments of the present disclosure may be embodied in many alternate forms and should not be construed as limited to exemplary embodiments of the present disclosure set forth herein. While describing the respective drawings, like reference numerals designate like elements.

It will be understood that although the terms “first,” “second,” etc. may be used herein to describe various components, these components should not be limited by these terms. These terms are used merely to distinguish one element from another. For example, without departing from the scope of the present disclosure, a first component may be designated as a second component, and similarly, the second component may be designated as the first component. The term “and/or” include any and all combinations of one of the associated listed items.

It will be understood that when a component is referred to as being “connected to” another component, it can be directly or indirectly connected to the other component. That is, for example, intervening components may be present. On the contrary, when a component is referred to as being “directly connected to” another component, it will be understood that there is no intervening components.

All terms including technical or scientific terms, unless being defined otherwise, have the same meaning generally understood by a person of ordinary skill in the art. It will be understood that terms defined in dictionaries generally used are interpreted as including meanings identical to contextual meanings of the related art, unless definitely defined otherwise in the present specification, are not interpreted as being ideal or excessively formal meanings.

In an exemplary embodiment according to the present disclosure, an EV charging system may be defined as a system for charging a high-voltage battery mounted within an EV using power of an energy storage device or a power grid of a commercial power source. The EV charging system may have various forms according to the type of EV. For example, the EV charging system may be classified into a conductive type using a charging cable and a non-contact wireless power transfer (WPT) type (also referred to as an ‘inductive type’). In an exemplary embodiment according to the present disclosure, a power source may include a residential or public electrical service or a generator utilizing vehicle-mounted fuel, and the like.

Terms used in the present disclosure are defined as follows.

“Electric Vehicle, EV”: An automobile, as defined in 49 CFR 523.3, intended for highway use, powered by an electric motor that draws current from an on-vehicle energy storage device, such as a battery, which is rechargeable from an off-vehicle source, such as residential or public electric service or an on-vehicle fuel powered generator. The EV may be four or more wheeled vehicle manufactured for use primarily on public streets, roads.

The EV may be referred to as an electric car, an electric automobile, an electric road vehicle (ERV), a plug-in vehicle (PV), a plug-in vehicle (xEV), etc., and the xEV may be classified into a plug-in all-electric vehicle (BEV), a battery electric vehicle, a plug-in electric vehicle (PEV), a hybrid electric vehicle (HEV), a hybrid plug-in electric vehicle (HPEV), a plug-in hybrid electric vehicle (PHEV), etc.

“Plug-in Electric Vehicle, PEV”: An Electric Vehicle that recharges the on-vehicle primary battery by connecting to the power grid.

“Plug-in vehicle, PV”: An electric vehicle rechargeable through wireless charging from an electric vehicle supply equipment (EVSE) without using a physical plug or a physical socket.

“Heavy duty vehicle; H.D. Vehicle”: Any four-or more wheeled vehicle as defined in 49 CFR 523.6 or 49 CFR 37.3 (bus).

“Light duty plug-in electric vehicle”: A three or four-wheeled vehicle propelled by an electric motor drawing current from a rechargeable storage battery or other energy devices for use primarily on public streets, roads and highways and rated at less than 4,545 kg gross vehicle weight.

“Wireless power charging system, WCS”: The system for wireless power transfer and control between the GA and VA including alignment and communications. This system transfers energy from the electric supply network to the electric vehicle electromagnetically through a two-part loosely coupled transformer.

“Wireless power transfer, WPT”: The transfer of electrical power from the AC supply network to the electric vehicle by contactless means.

“Utility”: A set of systems which supply electrical energy and may include a customer information system (CIS), an advanced metering infrastructure (AMI), rates and revenue system, etc. The utility may provide the EV with energy through rates table and discrete events. Also, the utility may provide information regarding certification on EVs, interval of power consumption measurements, and tariff.

“Smart charging”: A system in which EVSE and/or PEV communicate with power grid to optimize charging ratio or discharging ratio of EV by reflecting capacity of the power grid or expense of use.

“Automatic charging”: A procedure in which inductive charging is automatically performed after a vehicle is located in a proper position corresponding to a primary charger assembly capable of transferring power. The automatic charging may be performed after obtaining necessary authentication and access.

“Interoperability”: A state in which components of a system interwork with corresponding components of the system to perform operations aimed by the system. Also, information interoperability may refer to capability that two or more networks, systems, devices, applications, or components may efficiently share and easily use information without causing inconvenience to users.

“Inductive charging system”: A system transferring energy from a power source to an EV through a two-part gapped core transformer in which the two halves of the transformer, primary and secondary coils, are physically separated from one another. In the present disclosure, the inductive charging system may correspond to an EV power transfer system.

“Inductive coupler”: The transformer formed by the coil in the GA Coil and the coil in the VA Coil that allows power to be transferred with galvanic isolation.

“Inductive coupling”: Magnetic coupling between two coils. In the present disclosure, coupling between the GA Coil and the VA Coil.

“Ground assembly, GA”: An assembly on the infrastructure side including the GA Coil, a power/frequency conversion unit and GA controller as well as the wiring from the grid and between each unit, filtering circuits, housing(s) etc., necessary to function as the power source of wireless power charging system. The GA may include the communication elements necessary for communication between the GA and the VA.

“Vehicle assembly, VA”: An assembly within the vehicle including the VA Coil, rectifier/power conversion unit and VA controller as well as the wiring to the vehicle batteries and between each unit, filtering circuits, housing(s), etc., necessary to function as the vehicle part of a wireless power charging system. The VA may include the communication elements necessary for communication between the VA and the GA.

The GA may be referred to as a primary device (PD), and the VA may be referred to as a secondary device (SD).

“Primary device”: An apparatus which provides the contactless coupling to the secondary device. In other words, the primary device may be an apparatus extraneous to an EV. When the EV is receiving power, the primary device may act as the source of the power to be transferred. The primary device may include the housing and all covers.

“Secondary device”: An apparatus mounted within the EV which provides the contactless coupling to the primary device. In other words, the secondary device may be installed within the EV. When the EV is receiving power, the secondary device may transfer the power from the primary to the EV. The secondary device may include the housing and all covers.

“GA controller”: The portion of the GA which regulates the output power level to the GA Coil based on information from the vehicle.

“VA controller”: The portion of the VA that monitors specific on-vehicle parameters during charging and initiates communication with the GA to adjust output power level. The GA controller may be referred to as a primary device communication controller (PDCC), and the VA controller may be referred to as an electric vehicle communication controller (EVCC).

“Magnetic gap”: The vertical distance between the plane of the higher of the top of the litz wire or the top of the magnetic material in the GA Coil to the plane of the lower of the bottom of the litz wire or the magnetic material in the VA Coil when aligned.

“Ambient temperature”: The ground-level temperature of the air measured at the subsystem under consideration and not in direct sun light.

“Vehicle ground clearance”: The vertical distance between the ground surface and the lowest part of the vehicle floor pan.

“Vehicle magnetic ground clearance”: The vertical distance between the plane of the lower of the bottom of the litz wire or the magnetic material in the VA Coil mounted within a vehicle to the ground surface.

“VA Coil magnetic surface distance”: the distance between the plane of the nearest magnetic or conducting component surface to the lower exterior surface of the VA coil when mounted. This distance includes any protective coverings and additional items that may be packaged in the VA Coil enclosure. The VA coil may be referred to as a secondary coil, a vehicle coil, or a receive coil. Similarly, the GA coil may be referred to as a primary coil, or a transmit coil.

“Exposed conductive component”: A conductive component of electrical equipment (e.g. an electric vehicle) that may be touched and which is not normally energized but which may become energized in case of a fault.

“Hazardous live component”: A live component, which under certain conditions may output a harmful electric shock.

“Live component”: Any conductor or conductive component intended to be electrically energized in normal use.

“Direct contact”: Contact of persons with live components. (See IEC 61440)

“Indirect contact”: Contact of persons with exposed, conductive, and energized components made live by an insulation failure. (See IEC 61140)

“Alignment”: A process of detecting the relative position of primary device to secondary device and/or detecting the relative position of secondary device to primary device for the efficient power transfer that is specified. In the present disclosure, the alignment may direct to a fine positioning of the wireless power transfer system.

“Pairing”: A process by which a vehicle is correlated with the unique dedicated primary device, at which it is located and from which the power will be transferred. Pairing may include the process by which a VA controller and a GA controller of a charging spot are correlated. The correlation/association process may include the process of establishing a relationship between two peer communication entities.

“Command and control communication”: The communication between the EV supply equipment and the EV exchanges information necessary to start, control and terminate the process of WPT.

“High level communication (HLC)”: HLC is a particular type of digital communication. HLC is necessary for additional services which are not covered by command & control communication. The data link of the HLC may use a power line communication (PLC), but it is not limited.

“Low power excitation (LPE)”: LPE refers to a technique of activating the primary device for the fine positioning and pairing to allow the EV to detect the primary device, and vice versa.

“Service set identifier (SSID)”: SSID is a unique identifier consisting of 32-characters attached to a header of a packet transmitted on a wireless LAN. The SSID identifies the basic service set (BSS) to which the wireless device attempts to connect. The SSID distinguishes multiple wireless LANs. Therefore, all access points (APs) and all terminal/station devices that want to use a specific wireless LAN may use the same SSID. Devices that do not use a unique SSID are not able to join the BSS. Since the SSID is shown as plain text, it may not provide any security features to the network.

“Extended service set identifier (ESSID)”: ESSID is the name of the network to which one desires to connect. It is similar to SSID but may be a more extended concept.

“Basic service set identifier (BSSID)”: BSSID consisting of 48 bits is used to distinguish a specific BSS. In the case of an infrastructure BSS network, the BSSID may be medium access control (MAC) of the AP equipment. For an independent BSS or ad hoc network, the BSSID may be generated with any value.

The charging station may include at least one GA and at least one GA controller configured to manage the at least one GA. The GA may include at least one wireless communication device. The charging station may refer to a location having at least one GA, which is installed in home, office, public place, road, parking area, etc.

Additionally, it is understood that one or more of the below methods, or aspects thereof, may be executed by at least one controller. The term “controller” may refer to a hardware device that includes a memory and a processor. The memory may be configured to store program instructions, and the processor may be specifically programmed to execute the program instructions to perform one or more processes which are described further below. Moreover, it is understood that the below methods may be executed by an apparatus include the controller in conjunction with one or more other components, as would be appreciated by a person of ordinary skill in the art.

In exemplary embodiments according to the present disclosure, a light load driving or light load operation may include, for example, charging a high voltage battery with a charging voltage less than a predetermined rated voltage in the latter half of charging for the high voltage battery connected to the VA in the WPT system. Additionally, the light load operation may include a case in which the high-voltage battery of EV is charged at a relatively low voltage and at a low speed by using a low-speed charger such as a household charger.

Exemplary embodiments of the present disclosure are related to a method for facilitating wireless connection between an EV and a charging apparatus such as electric vehicle supply equipment (EVSE). Currently, much of the EV charging is performed in the conductive manner, but the inductive charging is also continuously being developed. Even in the case of using the conductive charging, data communications between the EV and the charging apparatus may be performed based on wireless communications.

Currently, standards for wireless charging are being developed to consider communications with the grid (i.e., vehicle-to-grid (V2G) communications) beyond the communications between the EV and the EVSE. For example, the international organization for standardization (ISO) 15118 (e.g., ISO 15118-2, 3 and 8) defines communication protocols for the wireless charging. The exemplary embodiments of the present disclosure may provide an optimal communication method for wireless charging.

Hereinafter, exemplary embodiments according to the present disclosure will be explained in detail by referring to accompanying figures.

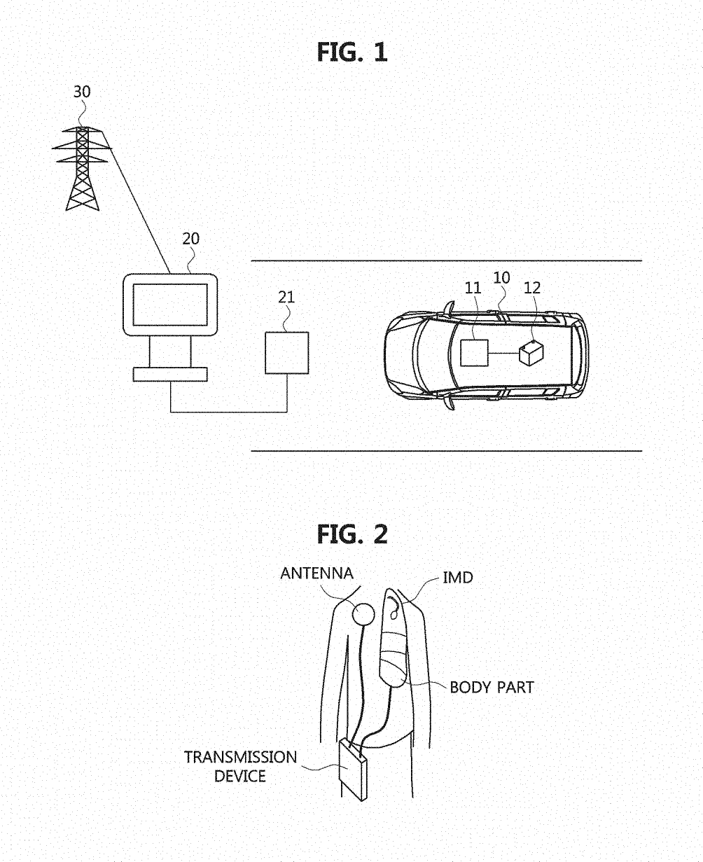

FIG. 1 is a conceptual diagram illustrating a concept of a wireless power transfer (WPT) to which exemplary embodiments of the present disclosure are applied. Referring to FIG. 1, a WPT may be performed by at least one component of an electric vehicle (EV) 10 and a charging station 20, and may be used for wirelessly transferring power to the EV 10. Particularly, the EV 10 may be usually defined as a vehicle that supplies an electric power stored in a rechargeable energy storage including a battery 12 as an energy source of an electric motor which is a power train system of the EV 10.

However, the EV 10 according to an exemplary embodiment of the present disclosure may include a hybrid electric vehicle (HEV) having an electric motor and an internal combustion engine together, and may include not only an automobile but also a motorcycle, a cart, a scooter, and an electric bicycle. Additionally, the EV 10 may include a power reception pad 11 that has a reception coil for charging the battery 12 wirelessly and may include a plug connection for conductively charging the battery 12. In particular, the EV 10 configured for conductively charging the battery 12 may be referred to as a plug-in electric vehicle (PEV).

The charging station 20 may be connected to a power grid 30 or a power backbone, and may provide an alternating current (AC) power or a direct current (DC) power to a power transmission pad 21 having a transmission coil via a power link. The charging station 20 may also be configured to communicate with an infrastructure management system or an infrastructure server that manages the power grid 30 or a power network via wired or wireless communications, and may be configured to perform wireless communications with the EV 10. The wireless communications may be Bluetooth, Zigbee, cellular, wireless local area network (WLAN), or the like. Additionally, the charging station 20 may be located at various places including a parking area attached to the owner's house of the EV 10, a parking area for charging an EV at a gas station or the like, a parking area at a shopping center or a workplace, but is not limited thereto.

A process of wirelessly charging the battery 12 of the EV 10 may begin with first disposing the power reception pad 11 of the EV 10 in an energy field generated by the power transmission pad 21 and causing the reception coil and the transmission coil to be interacted or coupled with each other. An electromotive force may be induced in the power reception pad 11 as a result of the interaction or coupling, and the battery 12 may be charged by the induced electromotive force.

The charging station 20 and the transmission pad 21 may be referred to as a ground assembly (GA) in whole or in part, where the GA may refer to the previously defined meaning. All or part of the internal components and the reception pad 11 of the EV 10 may be referred to as a vehicle assembly (VA), in which the VA may refer to the previously defined meaning.

Particularly, the power transmission pad or the power reception pad may be configured to be non-polarized or polarized. When a pad is non-polarized, there is one pole in a center of the pad and an opposite pole in an external periphery. A flux may be formed to exit from the center of the pad and return to external boundaries of the pad. When a pad is polarized, it may have a respective pole at either end portion of the pad. A magnetic flux may be formed based on an orientation of the pad.

FIG. 2 is a conceptual diagram illustrating a pacemaker as one of implantable medical devices to which exemplary embodiments of the present disclosure are applied. In FIG. 2, illustrated is a pacemaker implanted in a human body, which is a type of implanted medical devices (IMDs). A user may carry a transmission device (also referred to as a ‘medical telemetry device’) which is connected to the pacemaker and communicates with the outside. The transmission device may also include an antenna or may be connected with an antenna.

In particular, the IMD is a medical device implanted in a human body, may have a small computing platform of a programmable type that operates with a small battery to monitor a health condition of a patient (i.e., the user) or perform a medical therapy on the patient. For example, the IMD may include a deep brain neurostimulator, a gastric stimulator, a foot drop implant, a cochlear implant, a cardiac defibrillators, a cardiovascular defibrillator, a cardiovascular implantable electronic device (CIED), an insulin pumps, or the like. Among these, the CIED may include a pacemaker, an implantable cardioverter-defibrillator (ICD), a cardiac resynchronization therapy device (CRT), an implantable loop recorder (ILR), and an implantable cardiovascular monitor (ICM), but is not limited to the listed devices. The Federal Communications Commission (FCC) requires that an IMD, such as a pacemaker, uses a specified frequency to prevent interference from various information and communication devices.

Specifically, according to regulations of the FCC, a wireless medical telemetry service (WMTS) should be provided in a frequency range of about 608-614, 1395-1400 and/or 1427-1432 MHz. These frequency ranges may be used to remotely monitor the health condition of the patient. In addition to the WMTS, a medical telemetry device may operate at about 174-216 or 470-668 MHz, and a private land mobile device may operate at about 450-470 MHz.

Meanwhile, a society of automotive engineers (SAE) J2954, which is a standard document on wireless charging of EVs, regulates the exposure of electromagnetic fields and electromotive forces, since the IMDs may be influenced by the electromagnetic fields or electromotive forces emitted from the GA. The SAE J2954, a leading standard on wireless charging of EVs, establishes industrial standard guidelines that define interoperability, electromagnetic compatibility, minimum performances, safety, and acceptance criteria for testing for wireless charging of light duty plug-in EVs.

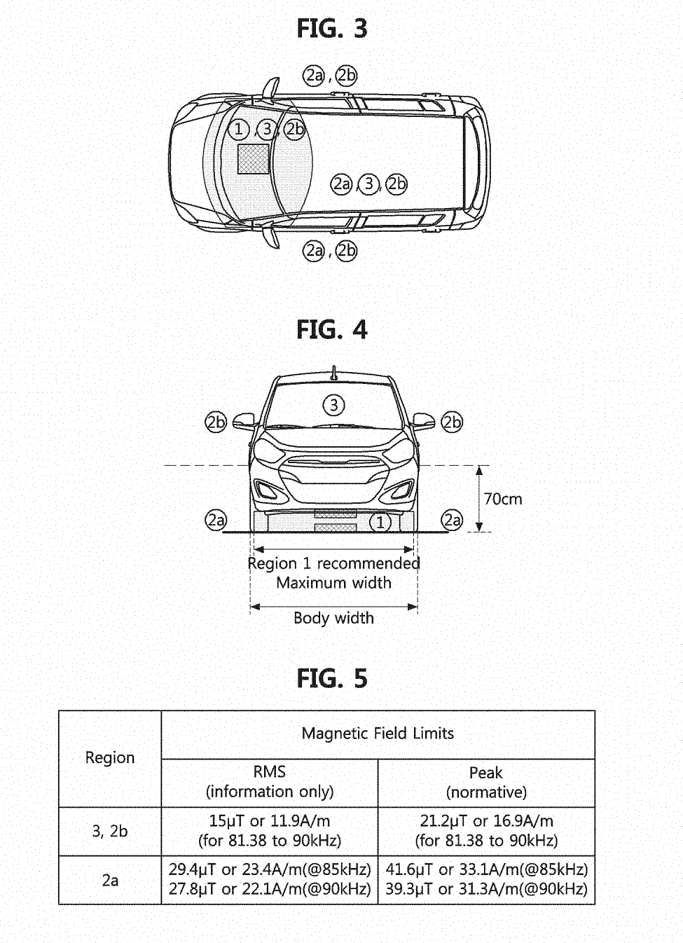

FIG. 3 is a plan view illustrating EMF regions to explain EMF limits, and FIG. 4 is a front view illustrating EMF regions to explain EMF limits. Referring to FIGS. 3 and 4, four physical regions may be defined to facilitate EMF safety management of a wireless charging system.

For example, region 1 may be an entire area underneath the vehicle, including and surrounding the wireless power assemblies (i.e., both of the VA and the GA). Region 1 shall not be extended beyond lower body structure edges (e.g., rocker panels or lower edges of bumpers). Additionally, region 2a may be a region around the vehicle, at heights less than about 70 cm above ground. Region 2b may additionally include areas under the vehicle. Region 2b may also be a region around the vehicle, at heights equal to or greater than about 70 cm above ground. Region 3 may be a vehicle interior.

The depicted shape and extent of region 1 is merely an example. The EMF management shall be applicable for all operational conditions such as a coupler offset or other system variations which may affect the worst case exposure. The boundaries of region 1 may be redefined for different systems, vehicle configurations, or operating conditions as long as EMF safety management principles and requirements are met for each configuration and condition.

FIG. 5 is a table listing magnetic field limits of a pacemaker/IMD for each region in vehicle interior and exterior. Typically, it is expected that pacemakers and implanted neuro-stimulators operate as designed in 81.38 to 90 kHz fields below 21.2 μT peak. Thus, in case of magnetic fields in regions 3 and 2b shown in FIGS. 3 and 4, the root mean square (RMS) thereof is preferably less than 15 μT or 11.9 A/m, and the peak value thereof is required to be less than 21.2 μT or 16.9 A/m in the range from 81.33 to 90 kHz.

Additionally, the magnetic field in region 2a may be preferably less than 29.4 μT or 23.4 A/m, based on RMS, at 85 kHz, and preferably less than 27.8 μT or 22.1 A/m at 90 kHz. The peak value is required to be less than 41.6 μT or 33.1 A/m at 85 kHz and 39.3 μT or 31.3 A/m at 90 kHz. Accordingly, exemplary embodiments of the present disclosure aim to provide a method for protecting a user of an IMD at the time of wirelessly charging an EV by preventing interference with other communication devices.

In other words, exemplary embodiments of the present disclosure provide a method in which an EV wireless charging system recognizes the IMD and protects the user wearing the IMD, when the user wearing the IMD operates an EV equipped with the EV wireless charging system to perform wireless charging, or when the user wearing the IMD is nearby the EV while the EV is being charged. Exemplary embodiments of the present disclosure may be applied to wireless charging of an EV in a parked state and also to wireless charging of an EV in a stopped state or in a traveling state.

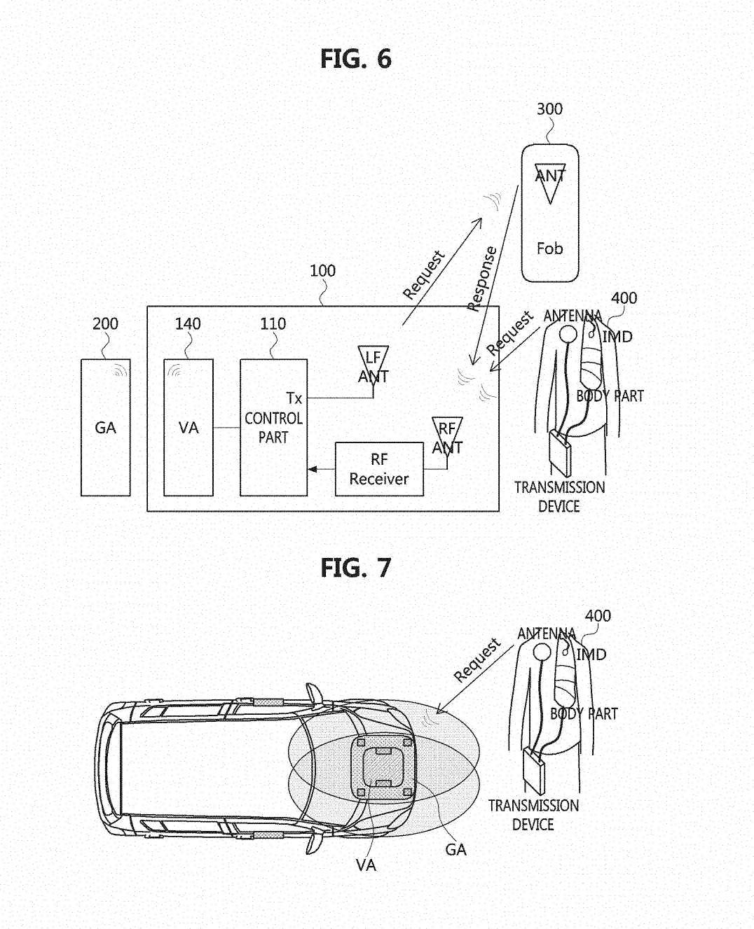

FIG. 6 is a conceptual diagram illustrating a wireless charging control system according to an exemplary embodiment of the present disclosure. Referring to FIG. 6, a wireless charging control system according to an exemplary embodiment of the present disclosure may include a vehicle 100, and a charging apparatus or a GA 200. The vehicle 100 may communicate with a smart key 300 and an IMD 400 worn by an occupant (or, driver or user). The vehicle 100 and components therein may also be operated by a controller within the vehicle.

Referring to FIG. 6, the vehicle may be configured to activate at least one LF magnetic field by using at least one LF antenna for position alignment between a transmission pad of the GA 200 and a reception pad of a VA. For example, when the VA 140 activates the at least one magnetic fields of the at least one LF antenna mounted on the vehicle, at least one LF receiver mounted on the GA may be configured to detect the at least one LF magnetic field to identify presence of the reception pad, and the GA may be configured to transmit information regarding the reception pad to the vehicle using an RF signal. The vehicle may be configured to receive and process the RF signal using an RF antenna and an RF receiver. The frequency band of the RF signal used may be about 433.92 MHz.

The RF communication scheme may also be used for communications between the vehicle 100 and the smart key 300, and may also be used for the smart key (or, fob) 300 to transmit a response to a request which is transmitted by the vehicle 100 via the LF antenna. In particular, a smart key system may use the LF or RF communication scheme to provide a passive entry function (i.e., door lock or unlock functions) for opening or closing a door or a trunk for the driver holding the smart key, and a passive engine starter function for starting an engine of the vehicle 100.

Moreover, the IMD 400 may be configured to periodically (e.g., at intervals of about 10 to 30 seconds) communicate with an external diagnostic device to thus monitor the operation of the IMD. The frequency band used herein may be, for example, the frequency band for MDRS as described above. In other words, the frequency band used for communications between the IMD and the external diagnostic device may be the same as or partially overlapped with the RF communication band used in the vehicle. Therefore, the signals that the IMD periodically transmits may be detectable by the RF antenna and receiver of the vehicle 100. In other words, the vehicle may include the RF antenna and the RF receiver for communication with the charging apparatus or the GA 200 and the smart key 300, and in the exemplary embodiments of the present disclosure, by using the RF antenna and the RF receiver, the presence of the IMD located in or around the vehicle may be detected.

In the exemplary embodiments of the present disclosure, when the vehicle detects the signal of the IMD, the vehicle may be configured to output a notification regarding the presence of the IMD to the charging apparatus to cause the charging apparatus to adjust a charging output supplied to the vehicle. The vehicle may be configured to periodically monitor whether the signal of the IMD is detected, and provide the notification to the charging apparatus when the signal of the IMD is no longer detected, thereby allowing the charging apparatus to reset the charging output to an original value to perform the charging.

Using the exemplary embodiments of the present disclosure, the signal of the IMD may be detected and the charging output of the EV wireless charging apparatus may be adjusted to protect the user of the IMD, according to the regulations of the electromagnetic fields or electromotive forces which are defined by the related standards. The charging control method according to an exemplary embodiment of the present disclosure may be a charging control method applied when a user (i.e., passenger or driver) of an IMD enters an EV.

Referring to FIG. 6, when the user of the IMD opens a door of the EV and enters the EV, the EV may be configured to perform discovery of a GA to determine whether a nearby GA is present. A control part 110 or component (e.g. a controller) of the EV may be configured to operate the LF transmitter to activate at least one LF magnetic field through the at least one LF antenna, and transmit an identifier (ID) of each LF antenna through each of the at least one activated LF magnetic field. When the vehicle 100 detects a response signal from the GA 200, the presence of the GA may be identified. The vehicle may also be configured to verify the presence of the IMD by detecting a request signal transmitted from the IMD.

When the vehicle receives a wireless charging (WPT) start command, a charging control may be performed based on the presence of the GA and the presence of the IMD. When the response signal from the GA and the signal of the IMD signal are detected, the VA may be configured to limit a charging output and provide a notification to the GA. A typical charging power may be defined as a charging power used for a typical or general charging process when any IMD is not present in or around the EV. A typical charging power may also be defined according to the society of automotive engineers (SAE) J2954, the International Organization for Standardization (ISO) 19363, or the International Electrotechnical Commission (IEC) 61980.

According to the exemplary embodiments of the present disclosure, the charging output may be limited to the level of about 10% to 80% of the typical charging power defined by these standards. For example, according to the SAE J2954, the ISO 19363, or the IEC 61980, the typical charging power may vary depending on a WPT power class and may be set, for example, to 3.3 kW, 7.7 kW, 11 kW, or the like. Thus, the charging output may be limited to 80% of the typical charging power of 3.3 kW, 7.7 kW, 11 kW, or the like. On the other hand, when the response signal from the GA is detected and the signal of the IMD is not detected, a normal charging may be performed. When the response signal from the GA is not detected and the signal of the IMD is detected, the VA may enter a sleep mode.

FIG. 7 is a conceptual diagram illustrating a charging control according to an exemplary embodiment of the present disclosure that may be applied when a user of an IMD is located in vicinity of a WPT system. In particular, FIG. 7 shows an exemplary embodiment in which the user of the IMD approaches the vehicle while the vehicle is being charged wirelessly. In particular, the vehicle may be configured to detect a signal from the IMD through the vehicle's RF antenna and receiver. When the signal of the IMD is determined to be detected, the controller may be configured to limit the charging output of the VA. Thereafter, when the signal of the IMD is no longer detected, the VA may be configured to release the limit on the charging output and return to a normal charging state.

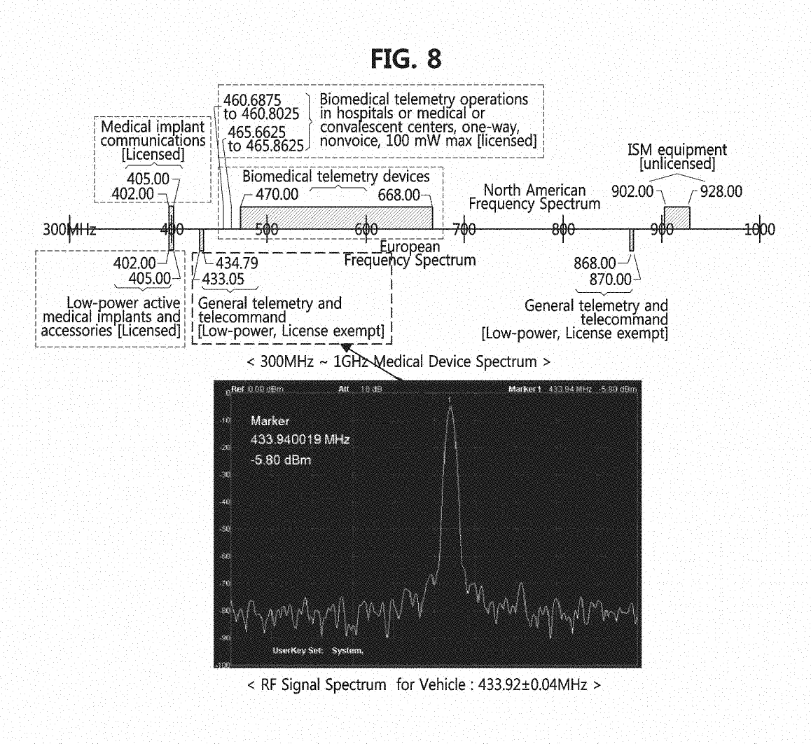

FIG. 8 is a diagram illustrating a frequency spectrum used for IMDs and a frequency spectrum of vehicle RF signals. Referring to FIG. 8, a frequency used by a medical device may have a range of about 300 MHz to 1 GHz. For medical implant communications of low-power active medical implants and accessories, a licensed frequency band of about 402 MHz to 405 MHz may be used. A low-power license-exempt frequency band of about 434.79 MHz to 433.05 MHz may be used for general telemetry and telecommand.

Additionally, a biomedical telemetry device may use a frequency range of about 470-668 MHz, and a general telemetry and telecommand device may use a frequency range of about 433.05-434.79 MHz. Biomedical telemetry operations in a hospital and telemetry operations at a recovery center may be performed at frequency ranges of about 460.6875-460.8025 MHz and 465.6625-465.8625 MHz, respectively.

Meanwhile, a frequency used for the vehicle RF signals may have a range of about 433.92±0.04 MHz, which is within the range of the frequency band used for the telemetry and telecommand of the medical device. Thus, since the frequency band of the RF telemetry used for the IMD to communicate with the external apparatus overlaps the frequency spectrum of the vehicle RF signals, the RF receiver of the vehicle according to the present disclosure may receive signals transmitted by the IMD in addition to the RF signals received from the GA or the smart key, and process the signals.

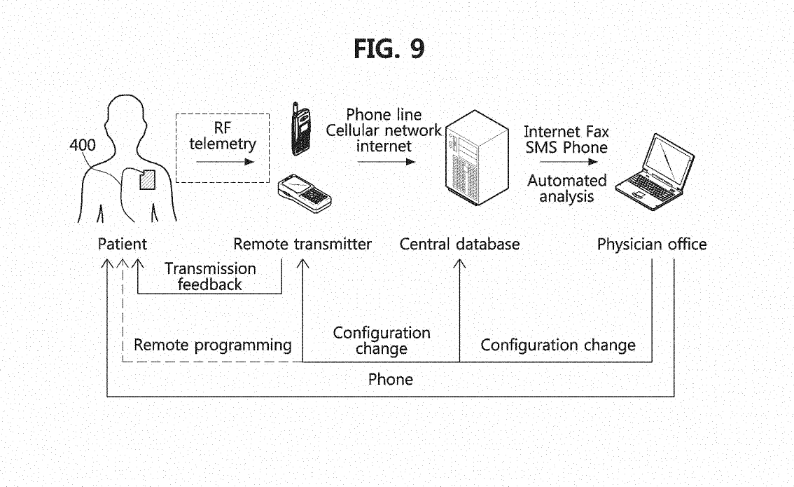

FIG. 9 is a conceptual diagram illustrating an IMD monitoring system. Referring to FIG. 9, the IMD 400 may be configured to store its own information, information regarding disease and treatment information for the patient, information regarding the patient, information related to an associated medical center, condition history of the patient, treatment history of the patient, and the like.

At least a portion of the information stored in the IMD may be transmitted to a central database located at the medical center or the like via a remote transmitter. In particular, communications using the RF telemetry device may be performed between the IMD and the remote transmitter. The EV or the charging control apparatus for the EV according to the present disclosure may be configured to detect a presence of the IMD by receiving an RF signal generated and transmitted periodically by the IMD. The information transferred to the central database may be provided to medical staff in the medical center or a separate physician office, and the medical staff may change prescription for the patient and perform a treatment action for the patient, if necessary, through analysis of the transferred information.

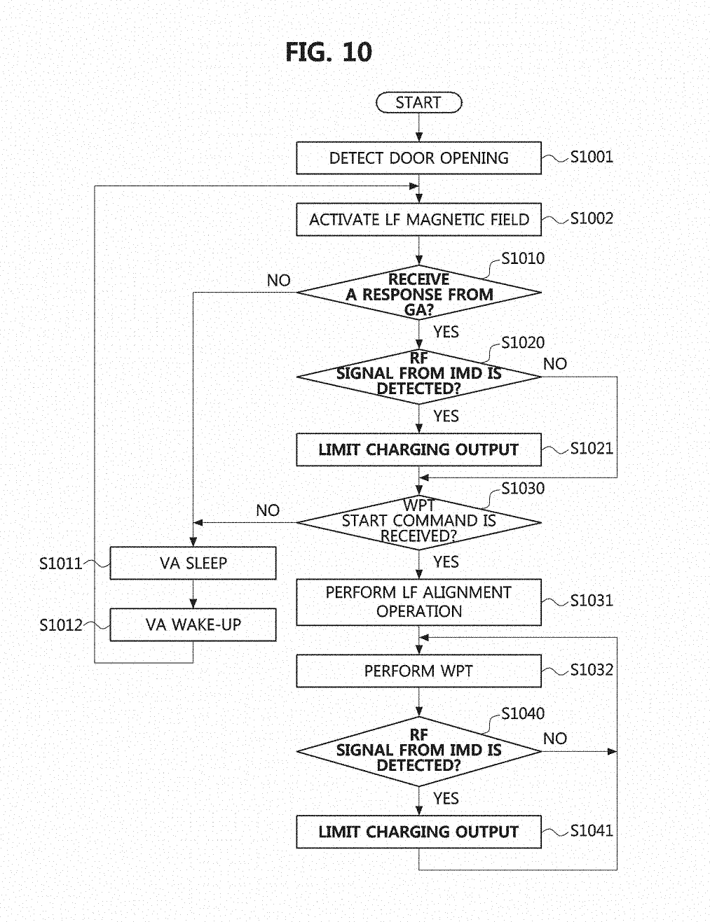

FIG. 10 is a flowchart illustrating a charging control method according to an exemplary embodiment of the present disclosure. The charging control method shown in FIG. 10 may be performed by at least one of the charging apparatus and the vehicle and in particular, by a controller thereof. Referring to FIG. 10, when a user (or, driver or passenger) enters an EV and intends to perform a wireless charging, a boarding operation may be performed after a door is opened. Thus, the vehicle may be configured to recognize the boarding of the user by detecting door opening (S1001).

The vehicle recognizing the boarding may then activate at least one LF magnetic field for discovering a GA (S1002). When the vehicle receives a response signal from a GA with respect to the at least one LF magnetic field (i.e., ‘YES’ in a step S1010) and detects an RF signal transmitted from an IMD (i.e., ‘YES’ in a step S1020), a charging output of the VA may be limited (S1021). Upon receipt of a WPT start command (i.e., ‘YES’ in a step S1030), an LF alignment operation may be performed to align transmission and reception pads (S1031), and wireless charging may be performed based on the limited charging output (S1032).

On the other hand, when the vehicle receives a response signal from a GA (i.e., ‘YES’ in the step S1010) but does not detect an RF signal transmitted from an IMD (i.e., ‘NO’ in the step S1020), upon receipt of a WPT start command, wireless charging may be performed without limiting the charging output (S1030, S1031, and S1032). When the vehicle does not detect a response signal from a GA (i.e., ‘NO’ in the step S1010) but detects an RF signal transmitted from the IMD (i.e., ‘YES’ in the step S1020), the VA may enter a sleep mode (S1011). The VA may wake up from the sleep mode when necessary (S1012), and repeat the above-described steps S1001 to S1030.

Additionally, in response to determining that the WPT start command is not received (i.e., ‘NO’ in the step S1030), the VA may enter the sleep mode (S1011). On the other hand, when the WPT start command is received, an LF alignment operation (S1031) and a wireless charging (i.e., WPT) operation (S1032) may proceed. When the vehicle detects an RF signal from an IMD during the wireless charging (i.e., ‘YES’ in a step S1040), the vehicle may be configured to recognize that a user wearing the IMD is passing around the vehicle and may be configured to limit the charging output of the VA (S1041). The operation of detecting the signal of the IMD may be periodically repeated, and when the signal from the IMD is no longer detected (i.e., ‘NO’ in the step S1040), the wireless charging (i.e., WPT) may be performed without limiting the charging output (S1032).

The operation sequence of the charging method illustrated in FIG. 10 is merely an example, and the steps of detecting the signal of the IMD signal, detecting the LF magnetic field, and the like may be performed at the same time, and the operation sequence of the subsequent steps may also be changed. Further, the IMD signal detection may be performed by the vehicle and a notification related thereto may be provided to the charging apparatus. Accordingly, the entity determining the limitation of the charging output may be the vehicle or the charging apparatus receiving relevant information from the vehicle.

Furthermore, when the charge control method illustrated in FIG. 10 is performed by an EV, such the charging control method may include detecting a signal transmitted from an IMD; determining a limitation on a charging output based on whether a signal is detected from an IMD; transmitting information related to the limitation on the charging output to the charging apparatus; and receiving power transferred from the charging apparatus. Additionally, the charging control method may include activating at least one LF magnetic field and detecting a response to the at least one LF magnetic field from the charging apparatus.

In particular, the signal transmitted from the IMD may be a RF signal. The information related to the limitation on the charging output may include information related to whether to limit the charging output and information related to a limitation ratio of the charging output to a typical charging power. The typical charging power may be defined according to at least one of the society of automotive engineers (SAE) J2954, the International Organization for Standardization (ISO) 19363, and the International Electrotechnical Commission (IEC) 61980.

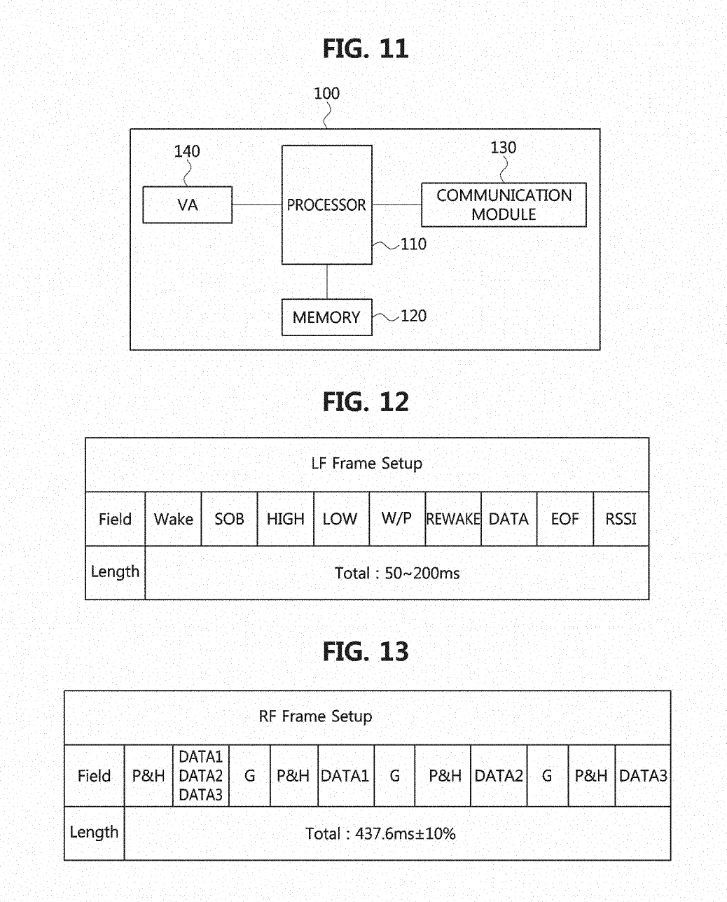

FIG. 11 is a block diagram illustrating an electric vehicle according to an exemplary embodiment of the present disclosure. Referring to FIG. 11, an EV 100 according to an exemplary embodiment of the present disclosure may include at least one processor 110 and a memory 120. The EV 100 may also include at least one communication module 130 and a VA 140.

The at least one communication module 130 may be at least one module configured to communicate with a charging apparatus, a smart key, and an IMD, and may include an LF communication module and an RF communication module. In particular, the LF communication module may include an LF antenna and may be configured to transmit an LF signal to the smart key. In other words, the LF communication module may be configured to activate an LF magnetic field. The RF communication module may include an RF antenna and may be configured to communicate with the smart key and the GA, and detect and process the RF signal transmitted by the IMD. The VA 140 is an in-vehicle charging module that includes a reception pad configured to receive power output through a transmission pad, and the reception pad may be coupled with the transmission pad of a charging apparatus.

Further, the memory 120 may be configured to store at least one instruction to be executed by the at least one processor, and the at least one instruction may be configured to cause the at least one processor to detect a presence of an IMD based on a signal input via the at least one communication module; determine a limitation on a charging output based on whether a signal from an IMD is detected; and transmit information related to the limitation on the charging output to the charging apparatus through the at least one communication module.

FIG. 12 is a diagram illustrating a structure of a frame used for LF communications applicable to exemplary embodiments of the present disclosure. As described referring to FIG. 6, the LF communication scheme may be utilized for bidirectional communications between the EV and the smart key together with the RF communication scheme.

Particularly, the LF communication scheme uses a transmission frequency band of about 125 KHz±0.5 KHz. The LF communication scheme may use pulse width modulation (PWM) and on-off keying (OOK) as a modulation scheme, and may have the frame structure shown in FIG. 12. One frame may have the length of about 50 to 200 ms, and the length of each field may vary depending on the application or function of the LF antenna.

FIG. 13 is a diagram illustrating a structure of a frame used for RF communications applicable to exemplary embodiments of the present disclosure. As described referring to FIG. 7, the RF communication scheme may be utilized for the bidirectional communications between the EV and the smart key together with the LF communication scheme. The RF communication scheme may also be a communication scheme used when the IMD transmits a signal to an external device. Therefore, the RF communication module of the EV may be configured to receive and process both the signals transmitted by the smart key and the signals transmitted by the IMD.

Particularly, the RF communication scheme uses a transmission frequency band of about 433.92 MHz±0.04 MHz. The RF communication scheme may use frequency shift keying (ASK) as a modulation scheme, and may have the frame structure shown in FIG. 13. One frame may have the length of about 437.6 ms±10%, and the length of each field may vary depending on the application or function of the RF antenna.

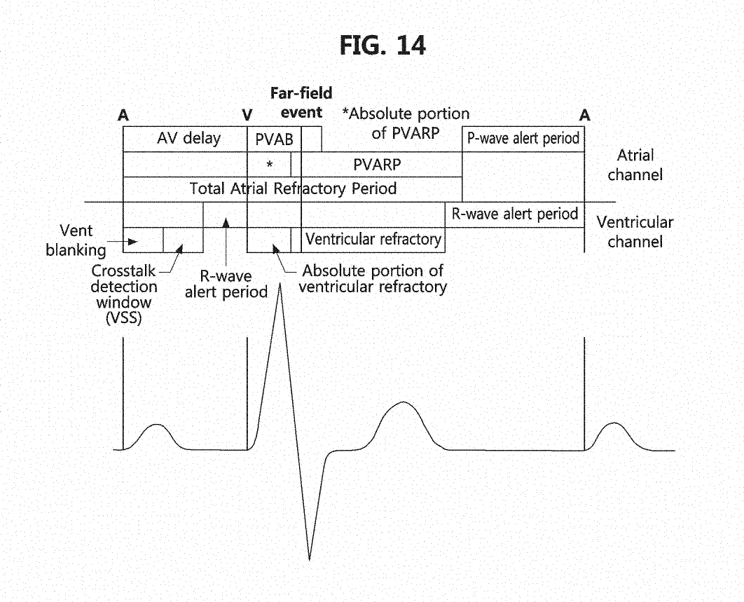

FIG. 14 is a diagram illustrating a timing cycle of a pacemaker applicable to exemplary embodiments of the present disclosure. Referring to FIG. 14, a pacemaker, which is an example of the IMD considered in the present disclosure, may have a basic interval, a basic rate, and a ventricular refractory period (VRP) as related time elements.

The basic interval may represent an interval between two ventricular pulses or two sensed ventricular events and may be determined according to the basic rate. In other words, the basic interval may be set to about 60.000/the basic rate. The VRP may be a period of generating a new beating signal immediately after a previous ventricular beating, and may be from about 200 ms to 250 ms. For example, when a signal of the type shown in FIG. 14 is detected through the RF communication module, the EV according to the present disclosure may be configured to determine that the signal has been generated and transmitted by the pacemaker.

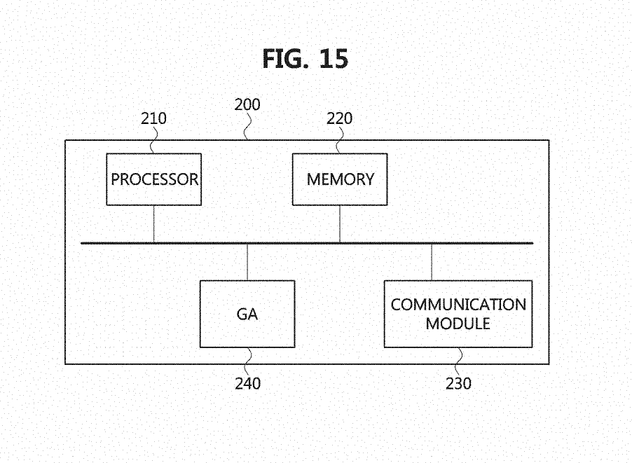

FIG. 15 is a block diagram illustrating a charging apparatus according to an exemplary embodiment of the present disclosure. Referring to FIG. 15, a charging apparatus 200 according to an exemplary embodiment of the present disclosure may include at least one processor 210 and a memory 220 storing at least one instruction to be executed by the at least one processor 210.

The charging apparatus 200 may also include a GA 240, and a communication module 230 configured to communicate with the EV. In particular, the communication module 230 may be configured to communicate with the EV using the RF communication scheme. The GA 240 is a power transmission module that includes a transmission pad coupled with a reception pad of the EV, and may be configured to supply power to the EV through the transmission pad.

The at least one instruction may be configured to cause the at least one processor 210 to detect a magnetic field activated by the EV; transmit a response signal to the magnetic field transmitted by the EV to the EV; receive from the EV information related to the limitation on the charging output set according to whether an IMD is present in or around the EV; and supply a power to the EV based on the information related to the limitation on the charging output. The magnetic field activated by the EV may be an LF magnetic field.

The methods according to exemplary embodiments of the present disclosure may be implemented as program instructions executable by a variety of computers and recorded on a non-transitory computer readable medium. The non-transitory computer readable medium may include a program instruction, a data file, a data structure, or a combination thereof. The program instructions recorded on the non-transitory computer readable medium may be designed and configured specifically for an exemplary embodiment of the present disclosure or can be publicly known and available to those who are skilled in the field of computer software.

Examples of the non-transitory computer readable medium may include a hardware device including ROM, RAM, and flash memory, which are configured to store and execute the program instructions. Examples of the program instructions include machine codes made by, for example, a compiler, as well as high-level language codes executable by a computer, using an interpreter. The above exemplary hardware device may be configured to operate as at least one software module to perform the operation of the present disclosure, and vice versa.

While some aspects of the present disclosure have been described in the context of an apparatus, it may also represent a description according to a corresponding method, wherein the block or apparatus corresponds to a method step or a feature of the method step. Similarly, aspects described in the context of a method may also be represented by features of the corresponding block or item or corresponding device. Some or all of the method steps may be performed by (or using) a hardware device such as, for example, a microprocessor, a programmable computer, or an electronic circuit. In various exemplary embodiments, one or more of the most important method steps may be performed by such an apparatus.

In exemplary embodiments, a programmable logic device (e.g., a field programmable gate array (FPGA)) may be used to perform some or all of the functions of the methods described herein. In addition, the FPGA may operate in conjunction with a microprocessor to perform one of the methods described herein. Generally, the methods are preferably performed by some hardware device.

For convenience in explanation and accurate definition in the appended claims, the terms “upper”, “lower”, “internal”, “outer”, “up”, “down”, “upper”, “lower”, “upwards”, “downwards”, “front”, “rear”, “back”, “inside”, “outside”, “inwardly”, “outwardly”, “internal”, “external”, “internal”, “outer”, “forwards”, and “backwards” are used to describe features of the exemplary embodiments with reference to the positions of such features as displayed in the figures.

The foregoing descriptions of specific exemplary embodiments of the present disclosure have been presented for purposes of illustration and description. They are not intended to be exhaustive or to limit the disclosure to the precise forms disclosed, and obviously many modifications and variations are possible in light of the above teachings. The exemplary embodiments were chosen and described to explain certain principles of the disclosure and their practical application, to enable others skilled in the art to make and utilize various embodiments of the present disclosure, as well as various alternatives and modifications thereof. It is intended that the scope of the disclosure be defined by the claims appended hereto and their equivalents

What is claimed is:

1. A charging control method performed by an electric vehicle (EV) receiving a power from a charging apparatus, comprising:
detecting, by a processor, a signal from an implanted medical device (IMD);
determining, by the processor, a limitation on a charging output based on whether the signal from the IMD is detected;
transmitting, by the processor, information related to the limitation on the charging output to the charging apparatus; and
receiving, by the processor, the power from the charging apparatus.

2. The charging control method according to claim 1, wherein the signal from the IMD is a radio frequency (RF) signal.

3. The charging control method according to claim 1, further comprising:
activating, by the processor, a low frequency (LF) magnetic field; and
detecting, by the processor, from the charging apparatus a response to the LF magnetic field.

4. The charging control method according to claim 3, wherein the determining of a limitation regarding a charging output includes:
determining, by the processor, to limit the charging output when the signal from the IMD and the response from the charging apparatus are detected.

5. The charging control method according to claim 1, wherein the information related to the limitation includes information related to whether to limit the charging output and information related a limitation ratio of the charging output.

6. The charging control method according to claim 5, wherein the limitation ratio of the charging output is defined as a ratio to a typical charging power.

7. The charging control method according to claim 6, wherein the typical charging power is defined according to at least one of the society of automotive engineers (SAE) J2954, the International Organization for Standardization (ISO) 19363, and the International Electrotechnical Commission (IEC) 61980.

8. An electric vehicle (EV) comprising at least one communication module, a charging module including a reception pad coupled with a transmission pad of a charging apparatus and receiving a power through the reception pad, at least one processor, and a memory storing at least one instruction executed by the at least one processor, wherein the at least one instruction is configured to cause the at least one processor to:
detect a signal from an implanted medical device (IMD);
determine a limitation on a charging output based on whether the signal from the IMD is detected; and
transmit information related to the limitation on the charging output to the charging apparatus.

9. The EV according to claim 8, wherein the signal from the IMD is a radio frequency (RF) signal.

10. The EV according to claim 8, wherein the at least one instruction is further configured to cause the at least one processor to:
activate a low frequency (LF) magnetic field; and
detect a response to the LF magnetic field from the charging apparatus.

11. The EV according to claim 10, wherein the at least one instruction is further configured to cause the at least one processor to:
determine to limit the charging output when the signal from the IMD and the response from the charging apparatus are detected.

12. The EV according to claim 8, wherein the information related to the limitation includes information related to whether to limit the charging output and information related to a limitation ratio of the charging output.

13. The EV according to claim 12, wherein the limitation ratio of the charging output is defined as a ratio to a typical charging power.

14. The EV according to claim 13, wherein the typical charging power is defined according to at least one of the society of automotive engineers (SAE) J2954, the International Organization for Standardization (ISO) 19363, and the International Electrotechnical Commission (IEC) 61980.

15. The EV according to claim 8, wherein the at least one communication module is configured to communicate with the charging apparatus using a RF communication scheme.

16. A charging apparatus comprising a communication module communicating with an electric vehicle (EV), a power transmission module including a transmission pad coupled with a reception pad of the EV and supplying a power to the EV through the transmission pad, at least one processor, and a memory storing at least one instruction executed by the at least one processor, wherein the at least one instruction is configured to cause the at least one processor to:
detect a magnetic field activated by the EV;
transmit a response signal to the magnetic field to the EV;
receive from the EV information related to a limitation on a charging output configured based on whether an implanted medical device (IMD) is present in or around the EV; and
supply a power to the EV based on the information related to the limitation on the charging output.

17. The charging apparatus according to claim 16, wherein the information related to the limitation includes information related to whether to limit the charging output and information related to a limitation ratio of the charging output.

18. The charging apparatus according to claim 17, wherein the limitation ratio of the charging output is defined as a ratio to a typical charging power.

19. The charging apparatus according to claim 18, wherein the typical charging power is defined according to at least one of the society of automotive engineers (SAE) J2954, the International Organization for Standardization (ISO) 19363, and the International Electrotechnical Commission (IEC) 61980.

20. The charging apparatus according to claim 16, wherein the magnetic field activated by the EV is a low frequency (LF) magnetic field, and the at least one communication module is configured to communicate with the EV using an RF communication scheme.
